(19) United States
(12) Patent Application Publication
(10) Pub. No.: US 2011/0110874 A1
(43) Pub. Date: May 12, 2011

(54) Cosmetic Compositions

(76) Inventors: Hidekazu Tanaka, Toyonaka (JP); Masafumi Karita, Kobe (JP); Satoko Uchikawa, Kobe (JP); Yujin Saito, Tokyo (JP); Akira Matsubara, Kobe (JP)

(21) Appl. No.: 13/008,734

(22) Filed: Jan. 18, 2011

Related U.S. Application Data
(62) Division of application No. 12/009,051, filed on Jan. 16, 2008, now abandoned.

(51) Int. Cl. A61K 8/73; A61Q 17/04; A61K 8/64; A61K 31/455; A61Q 19/00; A61Q 19/02

(52) U.S. Cl. 424/59; 514/18.8; 514/356; 514/54

(57) ABSTRACT

The present invention relates to a composition comprising from about 0.1% to about 10% of a hydrophobic structuring agent; from about 0.1% to about 10% of a hydrophilic surfactant; from about 0.05% to about 8% of a non-crosslinked water-soluble polymer; and water, wherein the composition has a viscosity above about 15,000 cps, and has a highest G′ below about 15,000 dyne/cm² and a highest tan δ above about 0.4 in the angular frequency range of from about 0.1 rad/sec to about 1 rad/sec. The present invention also relates to a cosmetic product comprising a) a composition comprising from about 0.1% to about 10% of a hydrophobic structuring agent, from about 0.1% to about 10% of a hydrophilic surfactant; from about 0.01% to about 5% of a non-crosslinked water soluble polymer and water, and b) a container which contains the composition, wherein the composition has a viscosity above 15,000 cps, and recovers its surface flatness to have a Sa below 80 within about 24 hours when its surface is distorted to have a Sa above about 150.

### CROSS REFERENCE TO RELATED APPLICATIONS

This application claims the benefit of U.S. application Ser. No. 12/009,051 filed on Jan. 16, 2008.

### FIELD OF THE INVENTION

The present invention relates to cosmetic compositions, cosmetic products, and preparation methods thereof, which provide consumer acceptable aesthetics, in particular, new usage experience.

### BACKGROUND OF THE INVENTION

A variety of products are available to consumers to provide skin care benefits. To be most effective, some products must be applied regularly and over an extended period of time. While delivery of specific skin care actives or compounds that can help to condition the skin and/or alleviate the damage caused by many extrinsic and intrinsic factors is of course important, to encourage frequent usage, it is also important that the product has a pleasant appearance and feel, both prior to and after application. For example, a product which provides brand new surface appearance during the use period can deliver to consumers a fresh feeling from the product appearance.

Consumers desiring more benefit and/or protection often tend to choose a thicker product. For example, a cream or gel composition tends to be perceived as offering greater skin benefits than a lotion. Creams or gels in general have a viscosity of above 15,000 cps, and show solid-like rheological behaviors. When a jar of cream or gel type product is opened for the first time, it has a nicely finished, aesthetically appealing smooth appearance. However, after the very first use, and every time thereafter that a consumer dips her fingers into the jar, the surface of the product remains with furrows and concavities.

Based on the foregoing, there is a need to provide cosmetic compositions with a thickness sufficient to convey an increased benefit, and which provide flat and shiny product appearance to deliver a fresh and pleasant feeling.

None of the existing art provides all of the advantages and benefits of the present invention.

### SUMMARY OF THE INVENTION

The present invention relates to a cosmetic composition comprising: from about 0.1% to about 10% of a hydrophobic structuring agent; from about 0.1% to about 10% of a hydrophilic surfactant; from about 0.01% to about 5% of a non-crosslinked water-soluble polymer; and water, wherein the composition has a viscosity above about 15,000 cps, and has a highest G′ below about 15,000 dyne/cm² and a highest tan δ above about 0.4 in the angular frequency range of from about 0.1 rad/sec to about 1 rad/sec.

The present invention also relates to a cosmetic product comprising: a) a composition comprising from about 0.1% to about 10% of a hydrophobic structuring agent; from about 0.1% to about 10% of a hydrophilic surfactant; from about 0.01% to about 5% of a non-crosslinked water soluble polymer; and water, and b) a container which contains the composition, wherein the composition has a viscosity above about 15,000 cps, and has a highest G′ below about 15,000 dyne/cm² and a highest tan δ above about 0.4 in the angular frequency range of from about 0.1 rad/sec to about 1 rad/sec.

The present invention also relates to a cosmetic product comprising: a) a composition comprising from about 0.1% to about 10% of a hydrophobic structuring agent; from about 0.1% to about 10% of a hydrophilic surfactant; from about 0.01% to about 5% of a non-crosslinked water soluble polymer; and water, and b) a container which contains the composition, wherein the composition has a viscosity above 15,000 cps, and recovers its surface flatness to have a Sa below 80 within about 24 hours when its surface is distorted to have a Sa above about 150.

The present invention also relates to a method of preparing compositions of the present invention comprising a) preparing an oil phase by mixing a hydrophobic structuring agent and a hydrophilic surfactant and optional ingredients for the oil phase, b) prepared an aqueous phase by mixing a water-soluble gum type polymer, water and optional ingredients for the oil phase, and c) dispersing the oil phase into the aqueous phase.

The present invention also relates to methods of using such compositions to regulate the condition of skin, said method comprising applying to the skin of a human in need of treatment.

These and other features, aspects, and advantages of the present invention will become evident to those skilled in the art from a reading of the present disclosure.

### BRIEF DESCRIPTION OF THE DRAWINGS

The patent or application file contains at least one photograph executed in color. Copies of this patent or patent application publication with color photographs will be provided by the Office upon request and payment of the necessary fee.

While the specification concludes with claims particularly pointing out and distinctly claiming the invention, it is believed that the present invention will be better understood from the following description of preferred, non-limiting embodiments and representations taken in conjunction with the accompanying drawings in which:

FIGS. 1A-D are color photographs of a preferably embodiment of the invention.

FIGS. 2A-D are color photographs of a comparative example.

FIG. 3 is plots of log oscillation frequency (x-axis) versus tan δ (y-axis) for preferably embodiments of the invention and comparative examples.

FIG. 4 is plots of log oscillation frequency (x-axis) versus G′(y-axis) for preferably embodiments of the invention and comparative examples.

### DETAILED DESCRIPTION

While the specification concludes with the claims particularly pointing and distinctly claiming the invention, it is believed that the present invention will be better understood from the following description.

All percentages and ratios used herein are by weight of the total composition and all measurements made are at 25° C., unless otherwise designated.

The term “cosmetic composition,” as used herein, refers to that suitable for topical application on mammalian keratinous tissue. Products contemplated by the term “cosmetic product” include, but are not limited to moisturizers, personal cleansing products, occlusive drug delivery patches, nail polish, powders, wipes, hair conditioners, skin treatment emulsions, shaving creams and the like.

The term “keratinous tissue,” as used herein, refers to keratin-containing layers disposed as the outermost protective covering of mammals (e.g., humans, dogs, cats, etc.) which includes, but is not limited to, skin, lips, hair, toenails, fingernails, cuticles, hooves, etc.

The term “skin care actives,” or “actives,” as used herein means compounds that, when applied to the skin, provide a benefit or improvement to the skin. It is to be understood that skin care actives are useful not only for application to skin, but also to hair, nails and other mammalian keratinous tissue.

The term “ambient conditions,” as used herein, refers to surrounding conditions under about one atmosphere of pressure, at about 50% relative humidity, and at about 25° C. unless otherwise specified.

The compositions of the present invention can include, consist essentially of, or consist of, the components of the present invention as well as other ingredients described herein.

The term “safe and effective amount,” as used herein, refers to an amount of a compound or composition sufficient to significantly induce a positive benefit, preferably a positive keratinous tissue appearance or feel benefit, or positive hair appearance or feel benefit, including independently or in combinations the benefits disclosed herein, but low enough to avoid serious side effects, i.e., to provide a reasonable benefit to risk ratio, within the scope of sound judgment of the skilled artisan.

The term “regulating skin condition,” as used herein, refers to improving skin appearance and/or feel, for example, by providing a benefit, such as a smoother appearance and/or feel. Herein, “improving skin condition” means effecting a visually and/or tactilely perceptible positive change in skin appearance and feel. The benefit may be a chronic benefit and may include one or more of the following: Reducing the appearance of wrinkles and coarse deep lines, fine lines, crevices, bumps, and large pores; thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); preventing loss of skin or hair elasticity, for example, due to loss, damage and/or inactivation of functional skin elastin, resulting in such conditions as elastosis, sagging, loss of skin or hair recoil from deformation; reduction in cellulite; change in coloration to the skin, hair, or nails, for example, under-eye circles, blotchiness (e.g., uneven red coloration due to, for example, rosacea), sallowness, discoloration caused by hyperpigmentation, etc.

The term “G′,” as used herein, refers an elastic (storage) modulus which is a measure of the amount of energy stored and retrieved when strain is applied to a composition.

The term “G″,” as used herein, refers to a viscous (loss) modulus which is a measure of the amount of energy dissipated as heat when strain is applied to a composition.

The term “tan δ,” as used herein, refers to a ratio of G″/G′ of a composition.

All percentages, parts and ratios are based upon the total weight of the skin care compositions of the present invention, unless otherwise specified. All such weights as they pertain to listed ingredients are based on the active level and, therefore, do not include carriers or by-products that may be included in commercially available materials, unless otherwise specified.

All publications cited herein are hereby incorporated by reference in their entirety.

The compositions of the present invention are useful for regulating the condition of skin and especially for regulating keratinous tissue condition.

The compositions of the present invention provide additional benefits, including stability, absence of significant (consumer-unacceptable) skin irritation and good aesthetics.

The composition of the present invention has a cream or gel appearance having a viscosity above 15,000 cps, preferably in the range of from about 20,000 cps to about 100,000 cps.

The composition of the present invention has a highest tan δ above about 0.4, preferably above about 0.5 in the angular frequency of from about 0.1 rad/sec to about 1 rad/sec. Tan δ of the composition may be inversely proportionate to an angular frequency in the angular frequency range of from about 0.1 rad/sec to about 1 rad/sec.

Each time that a portion of the composition of the present invention is removed from a container or the surface of the composition is disturbed or altered by, for example, a consumer's fingers or some other manual applicator, the composition in the container recovers flat and smooth surface and looks like the very first time the container is opened.

Without being bound by theory, the composition of the present invention has a weak gel network but still has a high viscosity enough to maintain cream or gel appearance, and it is believed that the weak gel-network allows flat surface recovery of the composition at ambient conditions after consumers' usage. The flat surface recovery is visibly observed. The perceptible recovery of flat surface may be characterized by Arithmetic Mean Deviation of the Surface (Sa) measured according to the Arithmetic Mean Derivative of the Surface Measurement provided in the Test Methods. Sa is a standard parameter, known in the art and defined as the arithmetic mean of the absolute values of the surface departures above and below the mean plan within the sampling area. The composition of the present invention, when it placed in a container, recovers its flat surface to have a Sa below about 80 within about 24 hours, preferably about 12 hours at ambient conditions when its surface is distorted to have a Sa above about 150 by, for example, taking out a portion of the composition by consumers' common usage. More preferably, the composition of the present invention recovers its surface flatness to have a Sa below about 40 within about 12 hours, at ambient conditions when its surface is distorted to have a Sa above about 150.

Since a product of the present composition can recover its flat surface after consumers' common usage, the consumers experience brand new product usage in every time they use the product.

The compositions of the present invention contain a hydrophobic structuring agent, a surfactant, a non-crosslinked water-soluble polymer, and water.

The compositions herein may also include a wide variety of other ingredients. The compositions of the present invention are described in detail hereinafter.

### Hydrophobic Structuring Agents

The composition of the present invention comprises at least one hydrophobic structuring agent. The hydrophobic structuring agent is present in the composition of the present invention at concentrations of from about 0.1% to about 10%, preferably from about 0.3% to about 6%, most preferably from about 0.5% to about 4% by weight.

The hydrophobic structuring agent of the present invention is selected from the group consisting of saturated C₁₆ to C₃₀ fatty alcohols, saturated C₁₆ to C₃₀ fatty alcohols containing from about 1 to about 5 moles of ethylene oxide, saturated C₁₆ to C₃₀ diols, saturated C₁₆ to C₃₀ monoglycerol ethers, saturated C₁₆ to C₃₀ hydroxy fatty acids, saturated C₁₆ to C₃₀ fatty acids, saturated C₁₆ to C₃₀ monoglycerol esters, saturated C₁₆ to C₃₀ acid monohexitol easters and mixtures thereof, having a melting point of at least about 40° C. A preferred hydrophobic structuring agent of the present invention is selected from the group consisting of saturated C₁₆ to C₃₀ fatty alcohols, saturated C₁₆ to C₃₀ fatty alcohols containing from about 1 to about 5 moles of ethylene oxide, saturated C₁₆ to C₃₀diols, saturated C₁₆ to C₃₀ monoglycerol ethers, saturated C₁₆ to C₃₀ hydroxy fatty acids, saturated C₁₆ to C₃₀ fatty acids, saturated C₁₆ to C₃₀ monoglycerol esters, saturated C₁₆ to C₃₀ acid monohexitol esters and mixtures thereof, having a melting point of at least about 40° C. Without being limited by theory, it is believed that these structuring agents are useful to assist in the formation of the rheological characteristic of the composition which contributes to the hydrolytic stability of the composition of the present invention. In particular structuring agents assist in the formation of the liquid crystalline gel network structures.

The preferred structuring agents of the present invention are selected from the group consisting of stearyl alcohol, cetyl alcohol, behenyl alcohol, stearic acid, palmitic acid, the polyethylene glycol ether of stearyl alcohol having an average of about 1 to about 5 ethylene oxide units, the polyethylene glycol ether of cetyl alcohol having an average of about 1 to about 5 ethylene oxide units, glycerol ester of stearic acid and mixtures thereof. More preferred structuring agents of the present invention are selected from the group consisting of stearyl alcohol, cetyl alcohol, behenyl alcohol, the polyethylene glycol ether of stearyl alcohol having an average of about 2 ethylene oxide units (steareth-2), the polyethylene glycol ether of cetyl alcohol having an average of about 2 ethylene oxide units, glycerol ester of stearic acid and mixtures thereof. Even more preferred structuring agents are selected from the group consisting of stearyl alcohol, cetyl alcohol, behenyl alcohol, and mixtures thereof.

### Surfactants

The composition of the present invention comprises at least one hydrophilic surfactant, preferably having a HLB above 10. The surfactant is present in the composition of the present invention at concentrations of from about 0.1% to about 10%, preferably from about 0.5% to about 5%, most preferably from about 1.0% to about 4% by weight.

Without being limited by theory, it is believed that the hydrophilic surfactant disperses the hydrophobic materials, such as structuring agents, in the water phase. The surfactant, at a minimum, must be hydrophilic enough to disperse in water. The exact surfactant chosen will depend upon the pH of the composition and the other components present.

A hydrophilic surfactant in the present invention can be selected from the group consisting of nonionic surfactants, anionic surfactants, cationic surfactants, amphoteric surfactants, and mixtures thereof.

One preferred for use herein are nonionic surfactants. Among the nonionic surfactants that are useful herein are those that can be broadly defined as condensation products of long chain alcohols, e.g. C8-30 alcohols, with sugar or starch polymers, i.e., glycosides. These compounds can be represented by the formula (S).ₙ—O—R wherein S is a sugar moiety such as glucose, fructose, mannose, and galactose; n is an integer of from about 1 to about 1000, and R is a C8-30 alkyl group. Examples of long chain alcohols from which the alkyl group can be derived include decyl alcohol, cetyl alcohol, stearyl alcohol, lauryl alcohol, myristyl alcohol, oleyl alcohol, and the like. Another useful nonionic surfactant includes the condensation products of sorbitol, with a fatty acid. Non-limiting examples include polysorbates such as Tweens available Uniqema, USA. Another useful non-ionic surfactant includes those that can be broadly defined as condensation products of fatty acids with sugar such as sucrose. Non-limiting examples of sugar with a fatty acid include the material given the CTFA designation sucrose cocoate, sold as a mixture with sorbitan stearate under the trade name Arlatone 2121 from Uniqema. Other useful nonionic surfactants include the condensation products of alkylene oxides with fatty acids (i.e. alkylene oxide esters of fatty acids). These materials have the general formula RCO(X).ₙ. OH wherein R is a C10-30 alkyl group, X is —OCH₂ CH₂— (i.e. derived from ethylene glycol or oxide) or —OCH₂CHCH₃— (i.e. derived from propylene glycol or oxide), and n is an integer from about 6 to about 100. Other nonionic surfactants are the condensation products of alkylene oxides with 2 moles of fatty acids (i.e. alkylene oxide diesters of fatty acids). These materials have the general formula RCO(X).ₙ OOCR wherein R is a C10-30 alkyl group, X is —OCH₂ CH₂— (i.e. derived from ethylene glycol or oxide) or —OCH_{2 CHCH}₃— (i.e. derived from propylene glycol or oxide), and n is an integer from about 6 to about 100. Other nonionic surfactants are the condensation products of alkylene oxides with fatty alcohols (i.e. alkylene oxide ethers of fatty alcohols). These materials have the general formula R(X)ₙOR′ wherein R is a C10-30 alkyl group, X is —OCH₂ CH₂— (i.e. derived from ethylene glycol or oxide) or —OCH₂ CHCH₃— (i.e. derived from propylene glycol or oxide), and n is an integer from about 6 to about 100 and R′ is H or a C10-30 alkyl group. Non-limiting examples of these alkylene oxide derived nonionic surfactants include ceteth-6, ceteth-10, ceteth-12, ceteareth-6, ceteareth-10, ceteareth-12, steareth-6, steareth-10, steareth-12, PEG-6 stearate, PEG-10 stearate, PEG-12 stearate, PEG-20 glyceryl stearate, PEG-80 glyceryl tallowate, PPG-10 glyceryl stearate, PEG-30 glyceryl cocoate, PEG-80 glyceryl cocoate, PEG-200 glyceryl tallowate, PEG-8 dilaurate, PEG-10 distearate, and mixtures thereof. Preferred among the nonionic surfactants are those selected from the group consisting of steareth-21, ceteareth-20, ceteareth-12, Tween-60, Tween-80, sucrose cocoate, steareth-100, PEG-100 stearate and mixtures thereof.

Another preferred surfactant herein is anionic surfactants. Non-limiting examples of anionic surfactants include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauroyl sarcosine, cocoyl sarcosine, ammonium cocoyl sulfate, sodium cocoyl sulfate, potassium cocoyl sulfate, monoethanolamine cocoyl sulfate, sodium tridecyl benzene sulfonate, sodium dodecyl benzene sulfonate, sodium cocoyl isethionate, and combinations thereof.

Another preferred surfactant herein is cationic surfactants. Non-limiting examples of cationic surfactants include quaternary ammonium salts or amido-amines having at least one fatty chain containing at least about 8 carbon atoms and mixtures thereof.

Another preferred surfactant herein is amphoteric surfactants. Non-limiting example of amphoteric surfactans includes phosphatidylcholine, hydrogenated phosphatidylcholine, lecithin, hydrogenated lecithin, hydroxylated lecithin, lysoletcithin and mixtures thereof.

### Non-Crosslinked Water-Soluble Polymers

The composition of the present invention comprises a non-crosslinked water-soluble polymer. The non-crosslinked water-soluble polymer is present in the composition of the present invention at concentrations of from about 0.01% to about 5%, preferably from about 0.1% to about 0.3%, most preferably from about 0.2% to about 2% by weight.

Without being bound by theory, a non-crosslinked water-soluble polymer may reduce viscosity of the composition and weaken gel-network of the composition which may contribute to the flat surface recovery property to the composition of the present invention.

A non-limiting class of non-crosslinked water-soluble polymers useful herein is gum type polymers. Non-limiting examples of gum type polymers useful herein include guar gum hydroxypropyl guar, locust been gum, gellan gum, natto gum, xanthan gum, and mixtures thereof.

Another non-limiting class of non-crosslinked water-soluble polymers useful herein is polysaccharide polymers. Non-limiting examples of polysaccharide polymers useful herein include those selected from cellulose, and cellulose derivatives such as carboxymethyl cellulose, hydroxyethyl cellulose, hydroxyethyl ethylcellulose, methylhydroxy ethylcellulose, hydroxypropyl cellulose, hydroxylpropyl methylcellulose, alkyl hydroxyalkyl cellulose ethers and mixtures thereof. Preferred among the alkyl hydroxyalkyl cellulose ethers are the material given the CTFA designation cetyl hydroxyethylcellulose, which is the ether of cetyl alcohol and hydroxyethylcellulose, sold under the tradename NATROSEL® CS PLUS from Aqualon Corporation (Wilmington, Del.). Other useful polysaccharides include sodium hyaluronate, and scleroglucans which are a linear chain of (1-3) linked glucose units with a (1-6) linked glucose every three units, a commercially available example of which is CLEAROGEL™ CS11 from Michel Mercier Products Inc. (Mountainside, N.J.).

### Water

The composition of the present invention comprises water. Water is present in the composition of the present invention at concentrations of from about 50% to about 95%, preferably from about 65% to about 90.

### Optional Ingredients

### Skin Care Actives

The compositions of the present invention may include at least one skin care active. Without being bound by theory, it is believed the present compositions provide versatility in formulating a variety of actives.

In any embodiment of the present invention, however, the actives useful herein can be categorized by the benefit they provide or by their postulated mode of action. However, it is to be understood that the actives useful herein can in some instances provide more than one benefit or operate via more than one mode of action. Therefore, classifications herein are made for the sake of convenience and are not intended to limit the active to that particular application or applications listed.

### Vitamin B₃ Compounds

Vitamin B₃ compound such as niacinamide is a preferred skin care active for use herein. The present invention preferably includes from about 0.1% to about 30%, more preferably from about 1% to about 20%, even more preferably from about 2% to about 10% of a vitamin B₃ compound.

As used herein, “vitamin B₃ compound” means a compound having the formula:

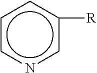

wherein R is —CONH₂ (i.e., niacinamide), —COOH (i.e., nicotinic acid) or —CH₂OH (i.e., nicotinyl alcohol); derivatives thereof; and salts of any of the foregoing. Exemplary derivatives of the foregoing vitamin B₃ compounds include nicotinic acid esters, including non-vasodilating esters of nicotinic acid (e.g., tocopheryl nicotinate), nicotinyl amino acids, nicotinyl alcohol esters of carboxylic acids, nicotinic acid N-oxide and niacinamide N-oxide.

### Whitening Agents

The present compositions may contain a whitening agent. The whitening agent useful herein refers to active ingredients that not only alter the appearance of the skin, but further improve hyperpigmentation as compared to pre-treatment. Useful whitening agents useful herein include ascorbic acid compounds, vitamin B₃ compounds, azelaic acid, butyl hydroxy anisole, gallic acid and its derivatives, hydroquinoine, kojic acid, arbutin, mulberry extract, undecylenoyl phenylalanine, cetyl pyridinium chloride, glycyrrhizic acid, tetrahydrocurcumin, and mixtures thereof. Use of combinations of whitening agents is also believed to be advantageous in that they may provide whitening benefit through different mechanisms.

When used, the compositions preferably contain from about 0.1% to about 10%, more preferably from about 0.2% to about 5%, by weight of the composition, of a whitening agent.

Ascorbic acid compounds are useful whitening agents. Preferably, the ascorbic acid compound useful herein is an ascorbate, ascorbyl esters of fatty acids, and ascorbic acid derivatives, for example, ascorbyl phosphates such as magnesium ascorbyl phosphate and sodium ascorbyl phosphate, ascorbyl sorbate, and ascorbyl glucoside.

Undecylenoyl hhenylalanine is the substituted amino acid that is also suitable for use herein as a whitening agent. It is available under the trade name Sepiwhite Msh, from Seppic.

Cetyl pyridinium chloride and tetrahydrocurcumin are also suitable for use herein as whitening agents.

Glycyrrhizic acid and glycyrrhetinic acid, a natural material derived from Glycyrrhiza Glabra, and its derivatives such as glycyrrhetinic acid are also suitable for use herein. Such materials are available from Maurzen or Ichimaru Pharcos.

### Peptides

Peptides, including but not limited to, di-, tri-, tetra-, and pentapeptides and derivatives thereof, may be included in the compositions of the present invention in amounts that are safe and effective. As used herein, “peptides” refers to both the naturally occurring peptides and synthesized peptides. Also useful herein are naturally occurring and commercially available compositions that contain peptides.

When included in the present compositions, peptides are preferably included in amounts of from about 1×10⁻⁶% to about 10%, more preferably from about 1×10⁻⁶% to about 0.1%, even more preferably from about 1×10⁻⁵% to about 0.01%, by weight of the composition.

### Sugar Amines

The compositions of the present invention may include a safe and effective amount of a sugar amine, which are also known as amino sugars. As used herein, “sugar amine” refers to an amine derivative of a six-carbon sugar.

Examples of sugar amines that are useful herein include glucosamine, N-acetyl glucosamine, mannosamine, N-acetyl mannosamine, galactosamine, N-acetyl galactosamine. Preferred for use herein is glucosamine. Additionally, combinations of two or more sugar amines may be used.

When included in the present compositions, the sugar amine is preferably included in amounts of from about 0.001% to about 20%, more preferably from about 1% to about 10%, even more preferably from about 2% to about 5%, by weight of the composition, of the sugar amine.

### Oil-Soluble Compounds

Optionally, the composition of the present invention can further comprise a oil-soluble compound. The oil-soluble compounds may be selected from oil-soluble vitamin compounds, oil-soluble terpene alcohols, phytosterol and derivatives thereof.

The amount of an oil-soluble compound may range from about 0.01% to about 10%, preferably, about 0.05% to about 5%, more preferably from about 0.1% to about 3%, by weight of the composition.

A number of vitamins known by those in the art for providing various skin benefits are oil-soluble and some or all of their derivatives are oil-soluble. Non-limiting examples of such oil-soluble vitamin compounds include retinoids, vitamin C (e.g. ascorbyl palmitate), vitamin D, vitamin K, vitamin E, and mixtures thereof. Preferred for use herein are retinoids, vitamin E, and mixtures thereof.

Oil-soluble terpene alcohols that are useful herein include farnesol, derivatives of farnesol, isomers of farnesol, geraniol, derivatives of geraniol, isomers of geraniol, phytantriol, derivatives of phytantriol, isomers of phytantriol, and mixtures thereof. Preferred for use herein is farnesol.

Phytosterol and derivatives thereof are known for providing skin lightening benefits. Non-limiting examples of oil-soluble phytosterol derivatives include β-sitosterol, campesterol, brassicasterol, lupenol, α-spinasterol, stigmasterol, their derivatives, and combinations thereof.

### Skin Conditioning Agent

Optionally, the composition of the present invention can further comprise a skin conditioning agent. These agents may be selected from humectants, exfoliants or emollients. The amount of skin condition agent may range from about 1% to about 60%, preferably from about 2% to about 50%, more preferably from about 5% to about 40%, by weight of the composition.

Humectants are polyhydric alcohols intended for moisturizing, reducing scaling and stimulating removal of built-up scale from the skin. Typical polyhydric alcohols include polyalkylene glycols and more preferably alkylene polyols and their derivatives. Illustrative are propylene glycol, dipropylene glycol, polypropylene glycol, polyethylene glycol, sorbitol, hydroxypropyl sorbitol, hexylene glycol, 1,3-butylene glycol, 1,2,6-hexanetriol, ethoxylated glycerin, propoxylated glycerin and mixtures thereof. Most preferably the humectant is glycerin.

Exfoliants according to the present invention may be selected from C2-C30 alpha-hydroxycarboxylic acids, beta-hydroxycarboxylic acids and salts of these acids. Most preferred are glycolic, lactic and salicylic acids and their ammonium salts.

When the conditioning agent is an emollient it may be selected from hydrocarbons, fatty acids, fatty alcohols and esters.

### Sunscreen Agents

The compositions of the subject invention may optionally contain a sunscreen agent. Suitable sunscreen agents may be organic or inorganic.

Inorganic sunscreen agents useful herein include the following metallic oxides; titanium dioxide, zinc oxide, zirconium oxide, iron oxide, and mixtures thereof.

Organic sunscreen agents useful herein include homosalate, octocrylene, 2-ethylhexyl-p-methoxycinnamate (commercially available as PARSOL MCX), phenyl benzimidazole sulfonic acid, 2-hydroxy-4-methoxybenzophenone (Benzophenone-3), 2-ethylhexyl-salicylate, and mixtures thereof.

When included in the present compositions, the sunscreens are preferably included in amounts of from about 0.1% to about 20%, preferably from about 0.5% to about 10%, more preferably from about 1% to about 5%, by weight of the composition. Exact amounts will vary depending upon the sunscreen or sunscreens chosen and the desired Sun Protection Factor (SPF).

### Other Optional Ingredients

A variety of additional ingredients can be incorporated into the compositions of the present invention. Non-limiting examples of these additional ingredients includes; particular materials to modify skin feel or appearance; anti-acne actives; oil-soluble beta-hydroxy acids such as salicylic acid and derivatives thereof; chelators; flavonoid compounds; anti-inflammatory agents; anti-cellulite agents; desquamation actives; anti-oxidant/radical scavengers; tanning actives; skin soothing or skin healing actives such as panthenoic acid derivatives (including panthenol, dexpanthenol, ethyl panthenol), aloe vera, allantoin, bisabolol, and dipotassium glycyrrhizinate; antimicrobial or antifungal actives.

### Composition Preparation

The compositions of the present invention are generally prepared by conventional preparation of cosmetic products. Such methods typically involve mixing of the ingredients in one or more steps to a relatively uniform state, with or without heating, cooling, application of vacuum, and the like.

In one embodiment, the method of the present invention comprises;
preparing an oil phase by mixing a hydrophobic structuring agent and a surfactant and optional ingredients for the oil phase;
prepared an aqueous phase by mixing a water-soluble gum type polymer, water and optional ingredients for the oil phase; and
dispersing the oil phase into the aqueous phase.

In another embodiment, the method of the present invention comprises;
preparing an oil phase by mixing a hydrophobic structuring agent and a surfactant and optional ingredients for the oil phase;
prepared an aqueous phase by mixing a water-soluble gum type polymer, water and optional ingredients for the oil phase;
preparing a skin active-containing phase by mixing a skin active, water and optional ingredients for the skin active-containing phase;
dispesing the oil phase into the aqueous phase; and
dispersing the skin active-containing phase into the mixture of the oil phase and the aqueous phase.

The topical compositions of the present invention may be formulated into a facial skin cosmetic, eye cosmetic, lip cosmetic, moisturizer, wrinkle soothing serum, lotion, skin facial mask, skin cream, skin gel, eye gel, eye cream, lip gel, lip cream, cosmetic, foundation, or any other commonly known skin product or treatment.

### Products for Topical use

In one preferred embodiment, the product of the present invention is an oil-in-water emulsion composition having a viscosity in the range of from about 20,000 to about 100,000 contained in a jar or the like for consumers to see the surface of the cream when they use the product. In this embodiment, the composition is a cream composition.

### Method of use

Applicants have found that the compositions of the present invention are useful in a variety of applications directed to enhancement of mammalian skin. The methods of use for the compositions disclosed and claimed herein include, but are not limited to: 1) methods of increasing the substantivity of a cosmetic to skin; 2) methods of moisturizing skin; 3) methods of improving the natural appearance of skin; 4) methods of applying a color cosmetic to skin; 5) methods of preventing, retarding, and/or treating wrinkles; 6) methods of providing UV protection to skin; 7) methods of preventing, retarding, and/or controlling the appearance of oil; 8) methods of modifying the feel and texture of skin; 9) methods of providing even skin tone; 10) methods of preventing, retarding, and/or treating the appear of spider vessels and varicose veins; 11) methods of masking the appearance of vellus hair on skin; and 12) methods of concealing blemishes and/or imperfections in human skin, including acne, age spots, freckles, moles, scars, under eye circles, birth marks, post-inflammatory hyperpigmentation, etc. Each of the methods discussed herein involve topical application of the claimed compositions to skin.

### Test Methods

### Arithmetic Mean Derivative of the Surface Measurement

Arithmetic Mean Derivative (Sa) is measured by a commercially available 3-D topometry like Primos-compact (GFMesstechnik GmbH, Berlin, Germany) at ambient conditions. Sa measurement is based on optical stripe projection technique that projects parallel stripe patterns onto the surface of the material to be measured (S. Jaspers, et al., Rapid in vivo measurement of the topography of human skin by active image triahgulation using a digital micromirror device, Skin Research and Technology, 5, 195-207, 1999). The roughness of the surface of a composition is calculated in terms of average roughness (Sa-value). Sa is a commonly used parameter for roughness and it is described as follows:

$$Sa = \frac{1}{MN} {\sum }_{j = 1}^{N} {\sum }_{i = 1}^{M} \vert η ( {x}_{i} , {y}_{j} ) \vert \qquad ( 1 )$$

Here, M is evaluation length, N is evaluation width, and η(xᵢ, yⱼ) is height profile function. The evaluation length and width are the total length and width along which the roughness evaluation is performed. The height profile function η(xᵢ, yⱼ) is the amplitude of the profile above or below the reference line which is given by the traversed profile in such a ways that the areas of the profile appearing above and below are equal. Thus, Sa is the sum of the area, which exists between the profile line and reference line, divided by the evaluation length.

### Viscosity Measurement

A product viscosity is measured by a commercially available viscometer like BROOKFIELD DV II+Viscometer with Helipath T-C bar type spindle (BROOKFIELD ENGINEERING LABORATORIES, INC.) at 5 rpm/min at 25° C.

### Oscillation Frequency Measurement

Tan δ and G′ are measured by dynamic oscillatory measurements using AR-G2 Stress Control Rheometer (TA Instruments Japan Inc.) or its equivalent. A sample composition is placed between an aluminum parallel palate of 40 mm diameter and a Peltier plate, controlled at a temperature of approximately 25° C. using a Peltier system or its equivalent. A sample of thickness of approximately is 1 mm A Dynamic Frequency Sweep is performed on the composition in the oscillation frequency mode (from 0.06283 rad/sec to 251 rad/sec) at an applied constant stress (10 dyne/cm²) within the linear viscoelastic response of the composition. In this measurement an oscillatory shear stress is imposed on the composition, and the corresponding shear response is measured. The stress is defined by a component in phase with the displacement (storage modulus, G′) and a component 90° out of phase (loss modulus, G″). Results are quoted as G′ and tan δ (a ratio of G″/G′) at various angular frequencies from 0.06283 rad/sec to 251 rad/sec.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

Compositions are prepared by conventional methods from the following components.

Viscosity of Examples and Comparative Examples were measured according to the Viscosity Measurement Oscillation Frequency Measurement.

| | | | | | |
|---|---|---|---|---|---|
| Ingredient | Ex. 1 | Ex. 2 | Ex. 3 | Ex. 4 | Ex. 5 |

Phase A

| | | | | | |
|---|---|---|---|---|---|
| Water | 18.69 | 62.20 | 59.70 | 43.75 | 42.44 |
| Arlatone 2121 ¹⁾ | 3.00 | 1.00 | 3.00 | — | — |
| Nikkomulese LH ²⁾ | — | — | — | 4.50 | 4.50 |
| Glycerin | 5.00 | 5.00 | 5.00 | 5.00 | 5.00 |
| Butylene Glycol | — | 4.00 | 4.00 | — | — |
| Pentylene Glycol | — | — | — | 3.00 | 3.00 |
| Xanthan Gum, 2% | 40.00 | — | — | 5.00 | 5.00 |
| Sodium | — | 1.50 | 1.00 | — | — |
| Hyaluronate | | | | | |
| Pemulen TR-2 ³⁾, 2% | — | — | — | — | — |
| Benzyl Alcohol | 0.20 | 0.20 | 0.20 | 0.20 | 0.20 |
| Methylparaben | 0.10 | 0.10 | 0.10 | 0.15 | 0.15 |
| Sodium Benzoate | — | — | — | — | — |
| EDTA -2NA | — | — | — | 0.10 | 0.10 |
| Timiron MP-1001 | — | — | — | — | — |
| Supersheen ⁴⁾ | | | | | |
| SperonL-1500 ⁵⁾ | — | — | — | — | — |
| Tospearl 145A ⁶⁾ | — | — | — | — | — |
| TiO₂ GLW75CAP-MP 75% ⁷⁾ | — | — | — | — | — |

Phase B

| | | | | | |
|---|---|---|---|---|---|
| Water | 10.00 | 10.00 | 10.00 | 10.00 | 10.00 |
| Ascorbyl | — | — | — | 2.00 | 2.00 |
| Glucoside | | | | | |
| Sodium Citrate | — | — | — | 0.20 | 0.20 |
| L-Arginine | — | — | — | 1.02 | 1.02 |
| Niacinamide | 2.00 | 2.00 | 2.00 | 5.00 | 5.00 |
| D-Panthenol | — | — | — | — | — |
| Aminocoat ⁸⁾ | — | — | — | — | — |
| Pellicer LC-30 ⁹⁾ | — | — | — | — | — |
| Na Metabisulfate | — | — | — | — | — |

Phase C

| | | | | | |
|---|---|---|---|---|---|
| De-ionized Water | 5.00 | — | — | — | 1.00 |
| Sepiwhite Msh ¹⁰⁾ | 1.00 | — | — | — | 0.20 |
| Triethanolamine | 0.51 | — | — | — | 0.11 |
| Amino | — | — | — | — | — |
| Methylpropanol | | | | | |

Phase D

| | | | | | |
|---|---|---|---|---|---|
| Myrj 59P ¹¹⁾ | 0.20 | 0.20 | 0.20 | — | — |
| Tetraglyn 5-SV ¹²⁾ | — | — | — | 0.5 | 0.5 |
| Rheodol 10SV ¹³⁾ | — | — | — | — | — |
| Arlamol E ¹⁴⁾ | — | — | — | — | — |
| Batyl Alcohol | — | — | — | 0.30 | 0.30 |
| Behenyl Alcohol | 2.00 | 0.50 | 0.50 | — | — |
| Steraryl Alcohol | 1.00 | 1.00 | 2.00 | 0.20 | 0.20 |
| Cetyl Alcohol | 1.00 | 1.00 | 1.00 | 0.80 | 0.80 |
| Stearic Acid | — | — | — | — | — |
| KSG-15 ¹⁵⁾ | — | — | — | 2.00 | 2.00 |
| Cyclomethicone | — | — | — | 8.00 | 8.00 |
| KF96A-6CS ¹⁶⁾ | 1.00 | 1.00 | 1.00 | — | — |
| Isohexadecane | 3.0 | 3.50 | 3.50 | — | — |
| Isopropyl | 2.30 | 2.80 | 2.80 | 2.40 | 2.40 |
| Isostearate | | | | | |
| Triethylhexanoin | 4.00 | 4.00 | 4.00 | 3.20 | 3.20 |
| Caprylic/Capric | — | — | — | 2.00 | 2.00 |
| Triglyceride | | | | | |
| Sucrose | — | — | — | 0.60 | 0.60 |
| Polycottonseedate | | | | | |
| Tocopheryl | — | — | — | — | — |
| Acetate | | | | | |

Phase E

| | | | | | |
|---|---|---|---|---|---|
| Sepigel 305 ¹⁷⁾ | — | — | — | — | — |
| Dow Corning DC | — | — | — | — | — |
| 1503 Fluid ¹⁸⁾ | | | | | |
| Fragrance | — | — | — | 0.08 | 0.08 |
| Viscosity (cps) | 41,000 | 98,800 | 54,080 | 44,760 | 36,300 |

| | | | | |
|---|---|---|---|---|
| Ingredient | Ex. 6 | C. 1 | C. 2 | C. 3 |

Phase A

| | | | | |
|---|---|---|---|---|
| Water | 31.85 | 30.25 | 51.49 | 38.46 |
| Arlatone 2121 ¹⁾ | — | — | 1.00 | 4.00 |
| Nikkomulese LH ²⁾ | 3.50 | 3.50 | — | — |
| Glycerin | 10.00 | 5.00 | 10.00 | 5.00 |
| Butylene Glycol | — | — | 2.00 | 3.00 |
| Pentylene Glycol | 2.00 | 2.00 | 2.00 | |
| Xanthan Gum, 2% | 3.00 | — | — | 10.00 |
| Sodium | — | — | — | — |
| Hyaluronate | | | | |
| Pemulen TR-2 ³⁾, 2% | — | 6.00 | — | — |
| Benzyl Alcohol | 0.20 | 0.20 | 0.25 | 0.20 |
| Methylparaben | 0.15 | 0.15 | — | 0.10 |
| Sodium Benzoate | — | — | — | 0.05 |
| EDTA -2NA | 0.10 | 0.10 | 0.10 | 0.10 |
| Timiron MP-1001 | 0.30 | — | — | — |
| Supersheen ⁴⁾ | | | | |
| SperonL-1500 ⁵⁾ | 1.00 | — | — | — |
| Tospearl 145A ⁶⁾ | — | 1.50 | — | — |
| TiO₂ GLW75CAP-MP 75% ⁷⁾ | 0.13 | — | 0.13 | — |

Phase B

| | | | | |
|---|---|---|---|---|
| Water | 16.00 | 13.00 | 10.00 | 13.00 |
| Ascorbyl | 2.00 | 2.00 | — | — |
| Glucoside | | | | |
| Sodium Citrate | 0.20 | 0.20 | — | — |
| L-Arginine | 1.02 | 1.02 | — | — |
| Niacinamide | 5.00 | 5.00 | 5.00 | 5.00 |
| D-Panthenol | — | — | 0.50 | — |
| Aminocoat ⁸⁾ | 2.00 | — | — | 2.00 |
| Pellicer LC-30 ⁹⁾ | 1.00 | — | — | 1.00 |
| Na Metabisulfate | — | — | — | 0.01 |

Phase C

| | | | | |
|---|---|---|---|---|
| De-ionized Water | — | 8.35 | 5.00 | — |
| Sepiwhite Msh ¹⁰⁾ | — | 1.00 | 1.00 | — |
| Triethanolamine | — | 0.65 | — | — |
| Amino | — | — | 0.30 | — |
| Methylpropanol | | | | |

Phase D

| | | | | |
|---|---|---|---|---|
| Myrj 59P ¹¹⁾ | — | — | 0.10 | — |
| Tetraglyn 5-SV ¹²⁾ | — | — | — | — |
| Rheodol 10SV ¹³⁾ | 0.50 | 0.50 | — | — |
| Arlamol E ¹⁴⁾ | — | — | — | 2.00 |
| Batyl Alcohol | 0.30 | — | — | — |
| Behenyl Alcohol | 0.68 | 0.40 | — | — |
| Steraryl Alcohol | 1.50 | 1.10 | 0.48 | 3.00 |
| Cetyl Alcohol | 1.19 | 0.70 | 0.72 | 1.00 |
| Stearic Acid | — | — | 0.10 | — |
| KSG-15 ¹⁵⁾ | 2.50 | 2.50 | — | 3.00 |
| Cyclomethicone | 4.40 | 8.40 | — | — |
| KF96A-6CS ¹⁶⁾ | 1.70 | 1.70 | — | 1.00 |
| Isohexadecane | 3.00 | — | 3.00 | 2.00 |
| Isopropyl | 2.20 | 2.20 | 1.33 | 3.50 |
| Isostearate | | | | |
| Triethylhexanoin | 2.50 | 2.50 | — | 2.50 |
| Caprylic/Capric | — | — | — | — |
| Triglyceride | | | | |
| Sucrose | — | — | 0.67 | — |
| Polycottonseedate | | | | |
| Tocopheryl | — | — | 0.50 | — |
| Acetate | | | | |

Phase E

| | | | | |
|---|---|---|---|---|
| Sepigel 305 ¹⁷⁾ | — | — | 2.25 | — |
| Dow Corning DC | — | — | 2.00 | — |
| 1503 Fluid ¹⁸⁾ | | | | |
| Fragrance | 0.08 | 0.08 | 0.08 | 0.08 |
| Viscosity (cps) | 43,320 | 45,560 | 29,920 | 13,800 |

¹⁾ Arlatone 2121 (Sorbitan stearate and Sucrose cocoate): Available from Uniqema
²⁾ Nikkomulese LH (Glycerin, Hydrogenated lecithin, Hydroxypropyl methylcellulose stearoxy ether, Squalane and Sodium methyl stearoyl taurate): Available from Nikko Chemicals
³⁾ Pemulen TR-2 (Acrylates/C10-30 Alkyl Acrylate Crosspolymer): Available from Noveon Inc.
⁴⁾ Timiron MP-1001 Supersheen (Mica and TiO₂): Available from EMD Chemicals Inc
⁵⁾ Speron L-1500 (Silica): Available from Presperse, Inc.
⁶⁾ Tospearl 145A (Polymethylsilsesquioxane): Available from GE Toshiba Silicone Co., Ltd.
⁷⁾ Titanium Dioxide GLW75CAP-MP 75% (TiO₂, Water and Glycerin): Available from Kobo Products Inc.,
⁸⁾ Aminocoat (Betaine): Available from Asahi Kasei Chemicals Co.
⁹⁾ Pellicer LC-30 (Sodium dilauramidoglutamide lysine and water): Available from Asahi Kasei Chemicals Co.
¹⁰⁾ Sepiwhite Msh (Undecylenoyl phenylalanine): Available from Seppic
¹¹⁾ Myrj 59P (PEG-100 Stearate): Available from Uniqema
¹²⁾ Tetraglyn 5-SV (Polyglyceryl-4 pentastearate): Available from Nikko Chemicals Co.
¹³⁾ Rheodol 10SV (Sorbitan stearate): Available from Kao Co.
¹⁴⁾ Arlamol E (PPG-15 stearyl ether): Available from Uniqema
¹⁵⁾ KSG-15 (Cyclopentasiloxane and Dimethicone/Vinyl Dimethicone Crosspolymer): Available from Shin-Etsu Chemical Co.,
¹⁶⁾ KF96A-6CS (Dimethicone): Available from Shin-Estu Chemical Co.
¹⁷⁾ Sepigel 305 (Polyaerylamide, C13-14 Isoparaffin and Laureth-7): Available from Seppic
¹⁸⁾ Dow Corning DC 1503 Fluid (Dimethicone and Dimethiconol): Available from Dow Corning Corporation

In separate suitable containers are added the ingredients of Phase A, Phase B, Phase C Phase D and Phase E, and each phase is mixed using a suitable mixer (e.g., Anchor blade, propeller blade, IKA T25) to obtain homogenized phases. When each phase is homogenous, slowly add Phase D to Phase A while mixing Phase A with a suitable mixer (e.g., Anchor blade, propeller blade, IKA T25). When batch is homogenous, slowly add Phase B, Phase C and Phase E to the obtained mixture of Phase A and Phase D while mixing the batch (e.g., Anchor blade, propeller blade, IKA T25). Maintain mixing until batch is uniform. Pour product into a jar.

Selected examples and comparative examples were tested to measure Sa according to the Arithmetic Mean Derivative of the Surface Measurement as presented in the Test Methods using a Primos-compact available from GFMesstechnik GmbH, Berlin, Germany. 30 g of each composition of Examples 1-4 and Comparative Examples 1-3 contained in a 30 g jar, respectively, was left at ambient conditions at least for 24 hours, and Sa of each sample was measured. Immediately after deforming the surface of each sample with fingers, Sa of each sample was measured. The samples were left at ambient conditions, and Sa of each sample was measured after 4 hours, 8 hours or 24 hours after the deformation.

| Sa | Ex. 1 | Ex. 2 | Ex. 3 | Ex. 4 | C. 1 | C. 2 | C. 3 |
|---|---|---|---|---|---|---|---|
| Before deformation | 54.95 | 37.72 | 24.23 | 9.89 | 123.06 | 49.20 | 33.70 |
| O hr after deformation | 313.57 | 220.82 | 135.68 | 180.03 | 202.15 | 200.68 | 39.40 |
| 4 hrs after deformation | — | — | — | 29.50 | — | — | — |
| 8 hrs after deformation | 103.25 | 43.82 | 22.70 | — | — | — | — |
| 24 hrs after deformation | 76.57 | 38.38 | 24.13 | 11.42 | 142.74 | 174.64 | 19.04 |

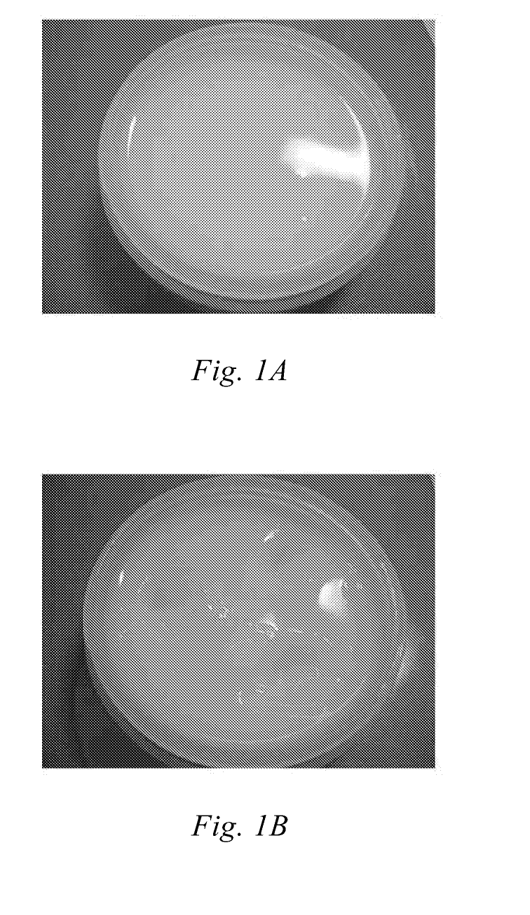

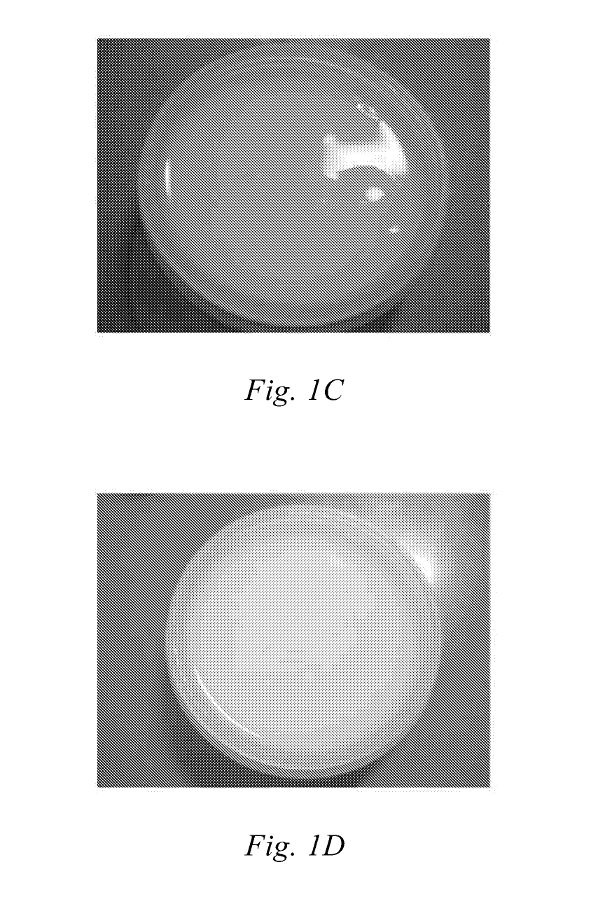

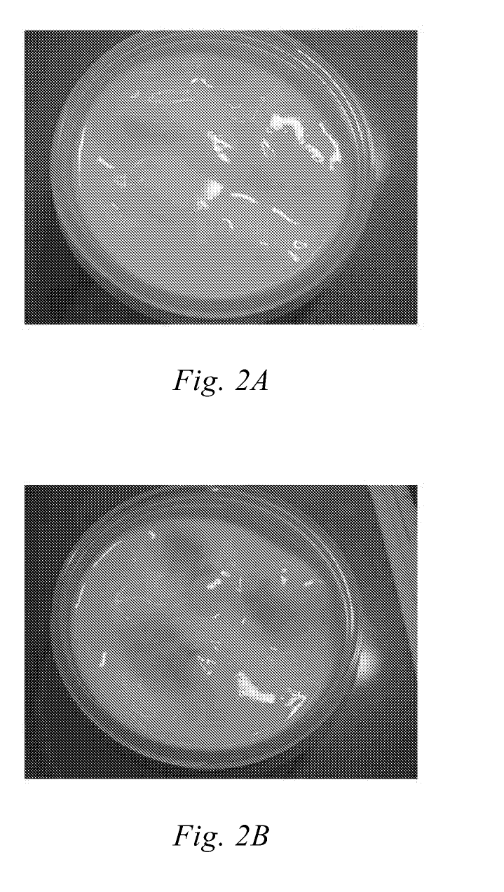

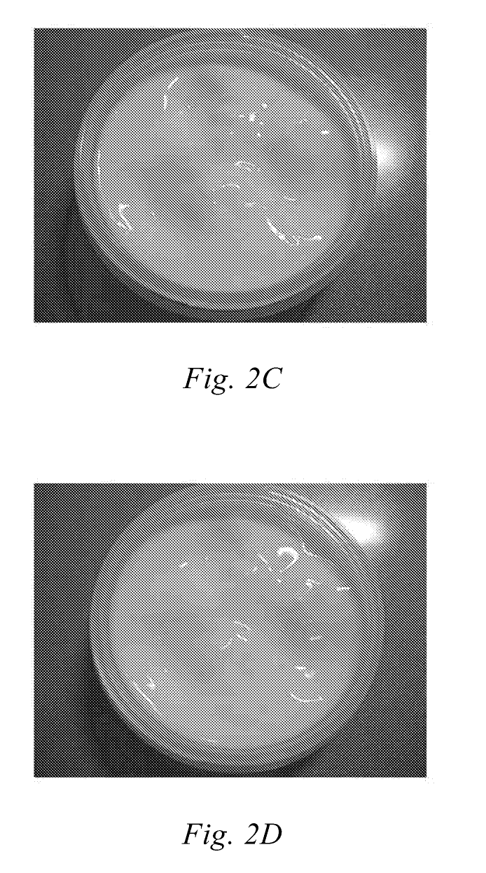

Photographs for Example 3 and Comparative Example 2 are provided as FIGS. 1A-D and 2A-D. FIGS. 1A-D are photographs of Example 3 taken before deformation, immediately after the deformation, and 8 and 24 hours after the deformation, respectively. FIGS. 2A-D are photographs of Comparative Example 2 taken as the same time points.

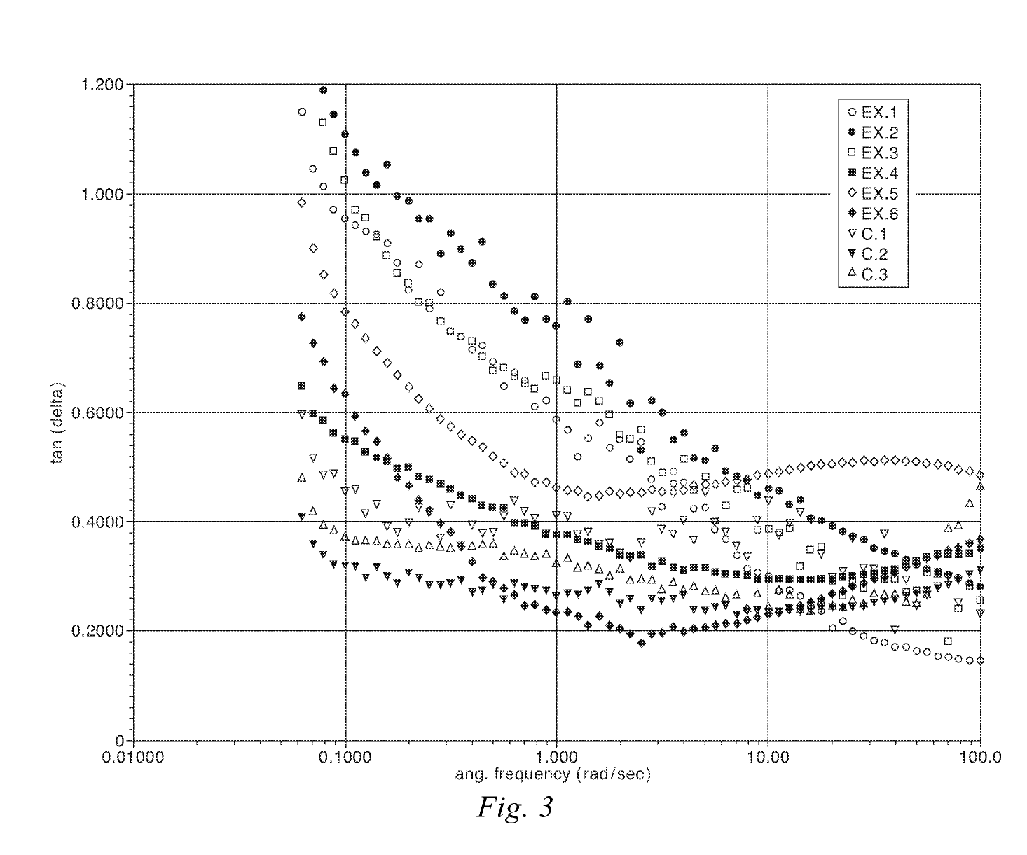

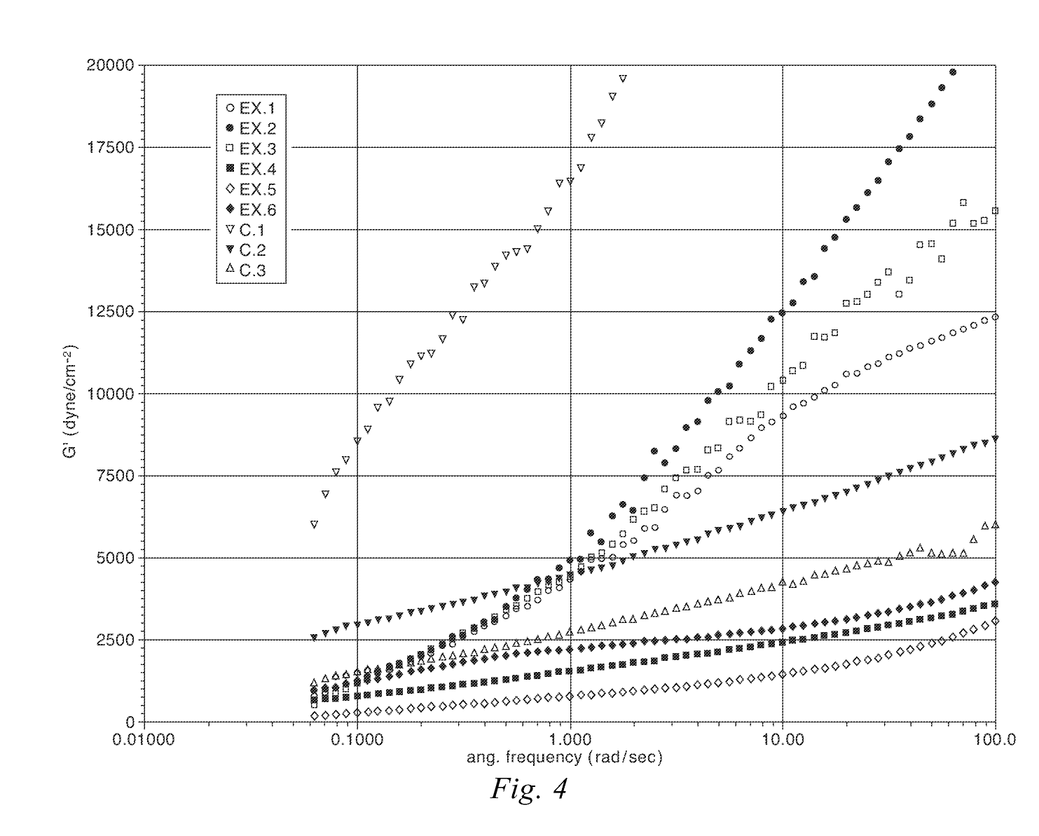

Examples and Comparative Examples were tested according to the Oscillation Frequency Measurement as presented in the Test Methods to determine tan δ and G′ of the compositions. AR-G2 was performed at the constant oscillation stress of 10 dyne/cm² at 25° C. Graphs of the resulting data of tan δ for the tested examples are provided in FIG. 3 and G′ data are provided in FIG. 4.

It is understood that the foregoing detailed description of examples and embodiments of the present invention are given merely by way of illustration, and that numerous modifications and variations may become apparent to those skilled in the art without departing from the spirit and scope of the invention; and such apparent modifications and variations are to be included in the scope of the appended claims.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention. What is claimed is:

1. A cosmetic composition comprising:
a) from about 0.1% to about 10% of a hydrophobic structuring agent;
b) from about 0.1% to about 10% of a hydrophilic surfactant;
c) from about 0.05% to about 8% of a non-crosslinked water-soluble polymer which is selected from the group consisting of gums, salts of hyaluronate and combinations thereof; and
d) water,
wherein the composition has a viscosity above about 15,000 cps, and has a highest G′ below about 15,000 dyne/cm² and a highest tan δ above about 0.4 in the angular frequency range of from about 0.1 rad/sec to about 1 rad/sec.

2. The composition according to claim 1, wherein said composition has a viscosity in the range of from about 20,000 cps to about 100,000 cps.

3. The composition according to claim 1, wherein said composition has a highest tan δ above about 0.5 in the angular frequency range of from about 0.1 rad/sec to about 1 rad/sec.

4. The composition according to claim 1, wherein said hydrophobic structuring agent is selected from the group consisting of saturated C₁₆ to C₃₀ fatty alcohols, saturated C₁₆ to C₃₀ fatty alcohols containing from about 1 to about 5 moles of ethylene oxide, saturated C₁₆ to C₃₀diols, saturated C₁₆ to C₃₀ monoglycerol ethers, saturated C₁₆ to C₃₀ hydroxy fatty acids, saturated C₁₆ to C₃₀ acid monohexitol esters and mixtures thereof, having a melting point of at least about 40° C.

5. The composition according to claim 1, wherein said hydrophilic surfactant is selected from the group consisting of non-ionic surfactants, anionic surfactants, cationic surfactants, amphoteric surfactants and mixtures thereof.

6. The composition according to claim 1, wherein said non-crosslinked water-soluble polymer is a gum type polymer.

7. The composition according to claim 1, wherein said composition is an oil-in-water type composition.

8. The composition according to claim 1, further comprising at least one compound selected from a group consisting skin care actives, skin conditioning agents, oil-soluble compounds, sunscreen agents and mixtures thereof.

9. The composition according to claim 8, wherein said skin care actives are selected from the group consisting of vitamin B₃ compounds, whitening actives, peptides, sugar amines and mixtures thereof.
